SEQUENCE LISTING (29 sequences)

SEQ ID NO: 1 - DNA, 1739 bp, Felis catus
ttactagaggatcctgcccacacatacatctcctccctccacagcccccaggcagttctgagaagcagcccagagaggcctgcggtgcctcctggaaaaggatgttagacgcagcccttccaccctgccctactgttgcggccacagcaggtataaaagggttccaggctggggagggagcacctgccactgcatcatgaagggggcttgtgttctcgtgcttctctgggctgccttgctcttgatctcgggtggaagtaggtgtctgggacatgagtgtctgggacacagattctccaggggttcaaacaccttcccagggcacttctgagcatggcgggaaggggaagggaagaatgtgtcctgatgaaggtctttcaaaagggagggtcagcttgtctttgtgttccagattgtgaaatttgcccagccgtgaagagggatgttgacctattcctgacgggaacccccgacgaatatgttgagcaagtggcacaatacaatgcactacctgtagtattggaaaatgccagaatactgaagaactgcgttgatgcaaaaatgacagaagaggataaggagaatgctctcagcgtgctggtgggtctagctctgtgtctgtgcctctgacgcctgtctggggggtctgctcagggcagtgcaggaggggggttgctcatgtttgttctccaccatggcccttccctgggaatctgggaggagaaagacgccatggctggggaagtagaggggcactcatgtggggcaagactcagcctacccctcaagctttggggctggcccaggctgctcaacgctgcttggccaccagcttggggggctgcaggccctcctatatccctggcatcacttggcctcagtgtcaggccctcagctctggccttcctgactccagcctctccagcacgtgagactggatcttcaaactgtttgcactagatgcttcctatctccaaacgtcagttccttttctcttaactcctcaagttccatattccacccccccccccaaaaaaaacctcatctgtcgtcattccctgggtcccagaggccattctgtgcctcaaatactgagagagaggaggaggggaggggaggggagaggagaggagaggagaggagaggagaggagaggagaggagaggagaggagaggagaggagaggagaggcagcttccaaaaagttctcctgccctgcccaggcctgggatgcctgagtggagaattccagtgaatcctctctctgctgtcccaaagtaggaacaagctactgcttcagcaacaagtgttcaaaggacagaagaaggaagcaggctggaccagctcattcctggagtctccagatgcccacaggtgcatctggagccctgccaggaccttcttgccagcgtctttctaaccaagtctaccacttctatccgagactgccctccatcccatcatagtcacccctcttcttcactctgtttcattggaggaagcttctaggcacaccctgggattctcttgttgtgcagtagattgggaagaaccaccttggcctgctcagatccagaagccaccctccaaacaagcctgcaggctcctccccacaaagtgtccagtgcgtgctcagtagtgtttgtccgttctcacgtacccctcaaggtctcaccaggtctcctgactttctctttgcaggacaaaatatacacaagtcctctgtgttaaaggtaact

SEQ ID NO: 2 - protein, 109 aa, Felis catus
MLDAALPPCPTVAATADEGGLCSRASLGCLALDLGWKDCEICPAVKRDVDLFLTGTPDEYVEQVAQYNALPVVLENARILKNCVDAKMTEEDKENALSVLDKIYTSPLC

SEQ ID NO: 3 - DNA, 2425 bp, Felis catus
cacatcctctccaagagctttgtcctcaagagtagaagggcttcccactcttaacagccaagggttgaggagccacccacatgtgccaggtccctgcccacaggcctttggagcttctggcggggggggggtgtgtgggctgggcttagggtgctagtagtttataaagcagcagaaatcctgtcctgagcagagcattctagcagctgacacgatgaggggggcactgcttgtgctggcattgctggtgacccaagcgctgggcgtcaagatgggtgagagcagatggagggacagaggaccttcctgatccttgccctgctctatctcactccttcacctcccatggtgatctccaaacaggttctagccacaaagttaagcggccatggggagatcattgtccaggagtcctgcagaacccccctgatgtttttagtcgttgaatggagggagaggtttggagatggaggggtcattagtcgtgcacacaataggggagagttagttgggggtagtggtgcttatttgaaaggcagaaacaggcaggctgggatgcccggagcaccggtcaggggtctctccggctgctctcttctgctgagagtgcctcatagaaaatgttccgtctgtctgggatgtaagcagtcctgggagtgggcaggtctccgcggaaggtgagtcagaagaccctggatatatgtgagttgctctcaagtggcgggcaaacaggaacctcctgctctgctgattcttttgtgaaggtgttttctgtttgtgtcttcagcggaaacttgccccattttttatgacgtcttttttgcggtggccaatggaaatgaattactgttggacttgtccctcacaaaagtcaatgctactgaaccagagagaacagccatgaaaaaaatccaggattgctacgtggagaacggactcatatccagggtcttggatggactagtcatggtaatttcctatccttccccgcctccccaaccttcacgttgcgcgtgcagcatattgtaatattccacatacagaccatgcagtcaggggctaatggcaggtaagagctataaacaatcgagcacataaacctttgctccgcgctctacagcacatagaatacgcaacctcacgccatgtgcacacccagcctgttcttctaccacacgtgtcccttgtgtgcgaattaccttacgcacagttggaaaataggggactaatatcggtgtggcatagaaagcgtgttgactcgtaggatttttttctttctaggttaggggtgtcagaattgcaggagtaggattttagccttccacaggaaagagaaagttcttcattcagctcctgcacatgtaggagccttgtcagttctagttgaggaatattgaaactaagcacctgccctcagactctcttcccaggaagggactccctggctttgggaagcttctggtttttggcttctgttttacttccccttgtgcccaccttgatggctgctattcctttggttcagagtctcacttccttctgtatcaattcagggtctaaagtcagatttccactctgtttgttctggtgcctgaggccctcgaggcagctcctagctacgtgcagctgcaccccagggctggtcagtgtatttctggtgaactatctttttctgttatttttcttgttgcacagttaggtcgattttggttagtctgtctcttacctctacttgccgttaagtgctgattctgtaaaatgagagctttgtgaagaagtggaatttcttgcatgactacgggcacccagggcacatgggattgttcacaacacacacatacacattccatacatccagtacacctgacagatgagtctcaggtgagggagacatcgcatggacccagactcagctaccttgcccctcacccaggcccatccccatcgcgccctccagaatcttctcctcttcttgcctcctcactggttgttcagactcctctggcacaggtgcgtgggtgacggggggggggggggggggcgtctccatcctggtctgactgatcgcggccctctctccagaaatcggtctgtgggctagaggttcttgctagggacggagcggaatcactggggatgaggcatgaggtgatcctgggggaatggatacgctgccatgcgctcaggtcttctgtccctcctcgtcttactctctcccagatagccatcaacgaatattgcatgggtgaagcagttcagaacaccgtagaagatctcaagctgaacactttggggagatgaatctttgccgctgatgccccttctgagccccatcctcctgtcctgttctttacacctaaagctggaatccagacacctgtcctcacctaattcactctcaatccaggctgactagaatctgcag

SEQ ID NO: 4 - protein, 73 aa, Felis catus
MRGALLVLALLVTQALGVKMETCPIFYDVFFAVANGNELLLDLSLTKVNATEPERTAMKKIQDCYVENGLISR

SEQ ID NO: 5 - DNA, 1739 bp, Felis catus
ttactagaggatcctgcccacacatacatctcctccctccacagcccccaggcagttctgagaagcagcccagagaggcctgcggtgcctcctggaaaaggatgttagacgcagcccttccaccctgccctactgttgcggccacagcaggtataaaagggttccaggctggggagggagcacctgccactgcatcatgaagggggctcgtgttctcgtgcttctctgggctgccttgctcttgatctcgggtggaagtaggtgtctgggacatgagtgtctgggacacagattctccaggggttcaaacaccttcccagggcacttctgagcatggcgggaaggggaagggaagaatgtgtcctgatgaaggtctttcaaaagggagggtcagcttgtctttgtgttccagattgtgaaatttgcccagccgtgaagagggatgttgacctattcctgacgggaacccccgacgaatatgttgagcaagtggcacaatacaatgcactacctgtagtattggaaaatgccagaatactgaagaactgcgttgatgcaaaaatgacagaagaggataaggagaatgctctcagcgtgctggtgggtctagctctgtgtctgtgcctctgacgcctgtctggggggtctgctcagggcagtgcaggaggggggttgctcatgtttgttctccaccatggcccttccctgggaatctgggaggagaaagacgccatggctggggaagtagaggggcactcatgtggggcaagactcagcctacccctcaagctttggggctggcccaggctgctcaacgctgcttggccaccagcttggggggctgcaggccctcctatatccctggcatcacttggcctcagtgtcaggccctcagctctggccttcctgactccagcctctccagcacgtgagactggatcttcaaactgtttgcactagatgcttcctatctccaaacgtcagttccttttctcttaactcctcaagttccatattccacccccccccccaaaaaaaacctcatctgtcgtcattccctgggtcccagaggccattctgtgcctcaaatactgagagagaggaggaggggaggggaggggagaggagaggagaggagaggagaggagaggagaggagaggagaggagaggagaggagaggagaggagaggcagcttccaaaaagttctcctgccctgcccaggcctgggatgcctgagtggagaattccagtgaatcctctctctgctgtcccaaagtaggaacaagctactgcttcagcaacaagtgttcaaaggacagaagaaggaagcaggctggaccagctcattcctggagtctccagatgcccacaggtgcatctggagccctgccaggaccttcttgccagcgtctttctaaccaagtctaccacttctatccgagactgccctccatcccatcatagtcacccctcttcttcactctgtttcattggaggaagcttctaggcacaccctgggattctcttgttgtgcagtagattgggaagaaccaccttggcctgctcagatccagaagccaccctccaaacaagcctgcaggctcctccccacaaagtgtccagtgcgtgctcagtagtgtttgtccgttctcacgtacccctcaaggtctcaccaggtctcctgactttctctttgcaggacaaaatatacacaagtcctctgtgttaaaggtaact

SEQ ID NO: 6 - protein, 109 aa, Felis catus
MLDAALPPCPTVAATADEGGSCSRASLGCLALDLGWKDCEICPAVKRDVDLFLTGTPDEYVEQVAQYNALPVVLENARILKNCVDAKMTEEDKENALSVLDKIYTSPLC

SEQ ID NO: 7 - DNA, 1739 bp, Felis catus
ttactagaggatcctgcccacacatacatctcctccctccacagcccccaggcagttctgagaagcagcccagagaggcctgcggtgcctcctggaaaaggatgttagacgcagcccttccaccctgccctactgttgcggccacagcaggtataaaagggttccaggctggggagggagcacctgccactgcatcatgaagggggcttgtgttctcgtgcttctctgggctgccttgctcttgatctggggtggaagtaggtgtctgggacatgagtgtctgggacacagattctccaggggttcaaacaccttcccagggcacttctgagcatggcgggaaggggaagggaagaatgtgtcctgatgaaggtctttcaaaagggagggtcagcttgtctttgtgttccagattgtgaaatttgcccagccgtgaagagggatgttgacctattcctgacgggaacccccgacgaatatgttgagcaagtggcacaatacaatgcactacctgtagtattggaaaatgccagaatactgaagaactgcgttgatgcaaaaatgacagaagaggataaggagaatgctctcagcgtgctggtgggtctagctctgtgtctgtgcctctgacgcctgtctggggggtctgctcagggcagtgcaggaggggggttgctcatgtttgttctccaccatggcccttccctgggaatctgggaggagaaagacgccatggctggggaagtagaggggcactcatgtggggcaagactcagcctacccctcaagctttggggctggcccaggctgctcaacgctgcttggccaccagcttggggggctgcaggccctcctatatccctggcatcacttggcctcagtgtcaggccctcagctctggccttcctgactccagcctctccagcacgtgagactggatcttcaaactgtttgcactagatgcttcctatctccaaacgtcagttccttttctcttaactcctcaagttccatattccacccccccccccaaaaaaaacctcatctgtcgtcattccctgggtcccagaggccattctgtgcctcaaatactgagagagaggaggaggggaggggaggggagaggagaggagaggagaggagaggagaggagaggagaggagaggagaggagaggagaggagaggagaggcagcttccaaaaagttctcctgccctgcccaggcctgggatgcctgagtggagaattccagtgaatcctctctctgctgtcccaaagtaggaacaagctactgcttcagcaacaagtgttcaaaggacagaagaaggaagcaggctggaccagctcattcctggagtctccagatgcccacaggtgcatctggagccctgccaggaccttcttgccagcgtctttctaaccaagtctaccacttctatccgagactgccctccatcccatcatagtcacccctcttcttcactctgtttcattggaggaagcttctaggcacaccctgggattctcttgttgtgcagtagattgggaagaaccaccttggcctgctcagatccagaagccaccctccaaacaagcctgcaggctcctccccacaaagtgtccagtgcgtgctcagtagtgtttgtccgttctcacgtacccctcaaggtctcaccaggtctcctgactttctctttgcaggacaaaatatacacaagtcctctgtgttaaaggtaact

SEQ ID NO: 8 - protein, 109 aa, Felis catus; identical to SEQ ID NO: 2

SEQ ID NO: 9 - DNA, 2425 bp, Felis catus
cacatcctctccaagagctttgtcctcaagagtagaagggcttcccactcttaacagccaagggttgaggagccacccacatgtgccaggtccctgcccacaggcctttggagcttctggcggggggggggtgtgtgggctgggcttagggtgctagtagtttataaagcagcagaaatcctgtcctgagcagagcattctagcagctgacacgatgaggggggcactgcttgtgctggcattgctggtgacccaagcgctgggcgtcaagatgggtgagagcagatggagggacagaggaccttcctgatccttgccctgctctatctcactccttcacctcccatggtgatctccaaacaggttctagccacaaagttaagcggccatggggagatcattgtccaggagtcctgcagaacccccctgatgtttttagtcgttgaatggagggagaggtttggagatggaggggtcattagtcgtgcacacaataggggagagttagttgggggtagtggtgcttatttgaaaggcagaaacaggcaggctgggatgcccggagcaccggtcaggggtctctccggctgctctcttctgctgagagtgcctcatagaaaatgttccgtctgtctgggatgtaagcagtcctgggagtgggcaggtctccgcggaaggtgagtcagaagaccctggatatatgtgagttgctctcaagtggcgggcaaacaggaacctcctgctctgctgattcttttgtgaaggtgttttctgtttgtgtcttcagcggaaacttgccccattttttatgacgtcttttttgcggtggccagtggaaatgaattactgttggacttgtccctcacaaaagtcaatgctactgaaccagagagaacagccatgaaaaaaatccaggattgctacgtggagaacggactcatatccagggtcttggatggactagtcatggtaatttcctatccttccccgcctccccaaccttcacgttgcgcgtgcagcatattgtaatattccacatacagaccatgcagtcaggggctaatggcaggtaagagctataaacaatcgagcacataaacctttgctccgcgctctacagcacatagaatacgcaacctcacgccatgtgcacacccagcctgttcttctaccacacgtgtcccttgtgtgcgaattaccttacgcacagttggaaaataggggactaatatcggtgtggcatagaaagcgtgttgactcgtaggatttttttctttctaggttaggggtgtcagaattgcaggagtaggattttagccttccacaggaaagagaaagttcttcattcagctcctgcacatgtaggagccttgtcagttctagttgaggaatattgaaactaagcacctgccctcagactctcttcccaggaagggactccctggctttgggaagcttctggtttttggcttctgttttacttccccttgtgcccaccttgatggctgctattcctttggttcagagtctcacttccttctgtatcaattcagggtctaaagtcagatttccactctgtttgttctggtgcctgaggccctcgaggcagctcctagctacgtgcagctgcaccccagggctggtcagtgtatttctggtgaactatctttttctgttatttttcttgttgcacagttaggtcgattttggttagtctgtctcttacctctacttgccgttaagtgctgattctgtaaaatgagagctttgtgaagaagtggaatttcttgcatgactacgggcacccagggcacatgggattgttcacaacacacacatacacattccatacatccagtacacctgacagatgagtctcaggtgagggagacatcgcatggacccagactcagctaccttgcccctcacccaggcccatccccatcgcgccctccagaatcttctcctcttcttgcctcctcactggttgttcagactcctctggcacaggtgcgtgggtgacggggggggggggggggggcgtctccatcctggtctgactgatcgcggccctctctccagaaatcggtctgtgggctagaggttcttgctagggacggagcggaatcactggggatgaggcatgaggtgatcctgggggaatggatacgctgccatgcgctcaggtcttctgtccctcctcgtcttactctctcccagatagccatcaacgaatattgcatgggtgaagcagttcagaacaccgtagaagatctcaagctgaacactttggggagatgaatctttgccgctgatgccccttctgagccccatcctcctgtcctgttctttacacctaaagctggaatccagacacctgtcctcacctaattcactctcaatccaggctgactagaatctgcag

SEQ ID NO: 10 - protein, 73 aa, Felis catus
MRGALLVLALLVTQALGVKMETCPIFYDVFFAVASGNELLLDLSLTKVNATEPERTAMKKIQDCYVENGLISR

SEQ ID NO: 11 - DNA, 2425 bp, Felis catus
cacatcctctccaagagctttgtcctcaagagtagaagggcttcccactcttaacagccaagggttgaggagccacccacatgtgccaggtccctgcccacaggcctttggagcttctggcggggggggggtgtgtgggctgggcttagggtgctagtagtttataaagcagcagaaatcctgtcctgagcagagcattctagcagctgacacgatgaggggggcactgcttgtgctggcattgctggtgacccaagcgctgggcgtcaagatgggtgagagcagatggagggacagaggaccttcctgatccttgccctgctctatctcactccttcacctcccatggtgatctccaaacaggttctagccacaaagttaagcggccatggggagatcattgtccaggagtcctgcagaacccccctgatgtttttagtcgttgaatggagggagaggtttggagatggaggggtcattagtcgtgcacacaataggggagagttagttgggggtagtggtgcttatttgaaaggcagaaacaggcaggctgggatgcccggagcaccagtcaggggtctctccggctgctctcttctgctgagagtgcctcatagaaaatgttccgtctgtctgggatgtaagcagtcctgggagtgggcaggtctccgcggaaggtgagtcagaagaccctggatatatgtgagttgctctcaagtggcgggcaaacaggaacctcctgctctgctgattcttttgtgaaggtgttttctgtttgtgtcttcagcggaaacttgccccattttttatgacgtcttttttgcggtggccaatggaaatgaattactgttggacttgtccctcacaaaagtcaatgctactgaaccagagagaacagccatgaaaaaaatccaggattgctacgtggagaacggactcatatccagggtcttggatggactagtcatggtaatttcctatccttccccgcctccccaaccttcacgttgcgcgtgcagcatattgtaatattccacatacagaccatgcagtcaggggctaatggcaggtaagagctataaacaatcgagcacataaacctttgctccgcgctctacagcacatagaatacgcaacctcacgccatgtgcacacccagcctgttcttctaccacacgtgtcccttgtgtgcgaattaccttacgcacagttggaaaataggggactaatatcggtgtggcatagaaagcgtgttgactcgtaggatttttttctttctaggttaggggtgtcagaattgcaggagtaggattttagccttccacaggaaagagaaagttcttcattcagctcctgcacatgtaggagccttgtcagttctagttgaggaatattgaaactaagcacctgccctcagactctcttcccaggaagggactccctggctttgggaagcttctggtttttggcttctgttttacttccccttgtgcccaccttgatggctgctattcctttggttcagagtctcacttccttctgtatcaattcagggtctaaagtcagatttccactctgtttgttctggtgcctgaggccctcgaggcagctcctagctacgtgcagctgcaccccagggctggtcagtgtatttctggtgaactatctttttctgttatttttcttgttgcacagttaggtcgattttggttagtctgtctcttacctctacttgccgttaagtgctgattctgtaaaatgagagctttgtgaagaagtggaatttcttgcatgactacgggcacccagggcacatgggattgttcacaacacacacatacacattccatacatccagtacacctgacagatgagtctcaggtgagggagacatcgcatggacccagactcagctaccttgcccctcacccaggcccatccccatcgcgccctccagaatcttctcctcttcttgcctcctcactggttgttcagactcctctggcacaggtgcgtgggtgacggggggggggggggggggcgtctccatcctggtctgactgatcgcggccctctctccagaaatcggtctgtgggctagaggttcttgctagggacggagcggaatcactggggatgaggcatgaggtgatcctgggggaatggatacgctgccatgcgctcaggtcttctgtccctcctcgtcttactctctcccagatagccatcaacgaatattgcatgggtgaagcagttcagaacaccgtagaagatctcaagctgaacactttggggagatgaatctttgccgctgatgccccttctgagccccatcctcctgtcctgttctttacacctaaagctggaatccagacacctgtcctcacctaattcactctcaatccaggctgactagaatctgcag

SEQ ID NO: 12 - protein, 73 aa, Felis catus; identical to SEQ ID NO: 4

SEQ ID NO: 13 - DNA, 2425 bp, Felis catus
cacatcctctccaagagctttgtcctcaagagtagaagggcttcccactcttaacagccaagggttgaggagccacccacatgtgccaggtccctgcccacaggcctttggagcttctggcggggggggggtgtgtgggctgggcttagggtgctagtagtttataaagcagcagaaatcctgtcctgagcagagcattctagcagctgacacgatgaggggggcactgcttgtgctggcattgctggtgacccaagcgctgggcgtcaagatgggtgagagcagatggagggacagaggaccttcctgatccttgccctgctctatctcactccttcacctcccatggtgatctccaaacaggttctagccacaaagttaagcggccatggggagatcattgtccaggagtcctgcagaacccccctgatgtttttagtcgttgaatggagggagaggtttggagatggaggggtcattagtcgtgcacacaataggggagagttagttgggggtagtggtgcttatttgaaaggcagaaacaggcaggctgggatgcccggagcaccggtcaggggtctctccggctgctctcttctgctgagagtgcctcatagaaaatgttccgtctgtctgggatgtaagcagtcctgggagtgggcaggtctccgcggaaggtgagtcagaagaccctggatatatgtgagttgctctcaagtggcgggcaaacaggaacctcctgctctgctgattcttttgtgaaggtgttttctgtttgtgtcttcagcggaaacttgccccattttttatgacgtcttttttgcggtggccaatggaaatgaattactgttggacttgtccctcacaaaagtcaatgctactgaaccagagagaacagccatgaaaaaaatccaggattgctacgtggagaacggactcatatccagggtcttggatggactagtcatggtaatttcctatccttccccgcctccccaaccttcacgttgcgcgtgcagcatattgtaatattccacatacagaccatgcagtcaggggctaatggcaggtaagagctataaacaatcgagcacataaacctttgctccgcgctctacagcacatagaatacgcaacctcacgccatgtgcacacccagcctgttcttctaccacacgtgtcccttgtgtgcgaattaccttacgcacagttggaaaataggggactaatatcggtgtggcatagaaagcgtgttgactcgtaggatttttttctttctaggttaggggtgtcagaattgcaggagtaggattttagccttccacaggaaagagaaagttcttcattcagctcctgcacatgtaggagccttgtcagttctagttgaggaatattgaaactaagcacctgccctcagactctcttcccaggaagggactccctggctttgggaagcttctggtttttggcttctgttttacttccccttgtgcccaccttgatggctgctattcctttggttcagagtctcacttccttctgtatcaattcagggtctaaagtcagatttccactctgtttgttctggtgcctgaggccctcgaggcagctcctagctatgtgcagctgcaccccagggctggtcagtgtatttctggtgaactatctttttctgttatttttcttgttgcacagttaggtcgattttggttagtctgtctcttacctctacttgccgttaagtgctgattctgtaaaatgagagctttgtgaagaagtggaatttcttgcatgactacgggcacccagggcacatgggattgttcacaacacacacatacacattccatacatccagtacacctgacagatgagtctcaggtgagggagacatcgcatggacccagactcagctaccttgcccctcacccaggcccatccccatcgcgccctccagaatcttctcctcttcttgcctcctcactggttgttcagactcctctggcacaggtgcgtgggtgacggggggggggggggggggcgtctccatcctggtctgactgatcgcggccctctctccagaaatcggtctgtgggctagaggttcttgctagggacggagcggaatcactggggatgaggcatgaggtgatcctgggggaatggatacgctgccatgcgctcaggtcttctgtccctcctcgtcttactctctcccagatagccatcaacgaatattgcatgggtgaagcagttcagaacaccgtagaagatctcaagctgaacactttggggagatgaatctttgccgctgatgccccttctgagccccatcctcctgtcctgttctttacacctaaagctggaatccagacacctgtcctcacctaattcactctcaatccaggctgactagaatctgcag

SEQ ID NO: 14 - protein, 73 aa, Felis catus; identical to SEQ ID NO: 4

SEQ ID NO: 15 - DNA, 18 bp, Artificial sequence
  primer
ctagaggatcctgcccac

SEQ ID NO: 16 - DNA, 21 bp, Artificial sequence
  primer
cgacgaatatgttgagcaagt

SEQ ID NO: 17 - DNA, 20 bp, Artificial sequence
  primer
cttcaaactgtttgcactag

SEQ ID NO: 18 - DNA, 20 bp, Artificial sequence
  primer
ctcaagttccatattccacc

SEQ ID NO: 19 - DNA, 21 bp, Artificial sequence
  primer
cttcttcactctgtttcattg

SEQ ID NO: 20 - DNA, 19 bp, Artificial sequence
  primer
caagtcctctgtgttaaag

SEQ ID NO: 21 - DNA, 20 bp, Artificial sequence
  primer
catcctctccaagagctttg

SEQ ID NO: 22 - DNA, 19 bp, Artificial sequence
  primer
gagaggtttggagatggag

SEQ ID NO: 23 - DNA, 13 bp, Artificial sequence
  primer
gtcttggatggac

SEQ ID NO: 24 - DNA, 17 bp, Artificial sequence
  primer
cacgttgcgcgtgcagc

SEQ ID NO: 25 - DNA, 20 bp, Artificial sequence
  primer
tcccaggaagggactccctg

SEQ ID NO: 26 - DNA, 17 bp, Artificial sequence
  primer
gtgcccaccttgatggc

SEQ ID NO: 27 - DNA, 18 bp, Artificial sequence
  primer
ggacccagactcagctac

SEQ ID NO: 28 - DNA, 19 bp, Artificial sequence
  primer
cttctcctcttcttgcctc

SEQ ID NO: 29 - DNA, 19 bp, Artificial sequence
  primer
caggctgactagaatctgc

(19) United States
(12) Patent Application Publication
(10) Pub. No.: US 2010/0062425 A1
(43) Pub. Date: Mar. 11, 2010

(54) CAT ALLERGEN

(75) Inventors: Chris Jones, Leicestershire (GB); Paul Jones, Nottingham (GB); Clive Tregaskes, Leicestershire (GB); Carina Salt, Leicestershire (GB)

Correspondence Address: FULBRIGHT & JAWORSKI, LLP, 1301 MCKINNEY, SUITE 5100, HOUSTON, TX, 77010-3095, US

(73) Assignee: MARS INCORPORATED, Mclean, VA (US)

(21) Appl. No.: 11/994,699

(22) PCT Filed: Jul. 6, 2006
(86) PCT No.: PCT/GB06/02503

§ 371 Date: Apr. 8, 2008

(30) Foreign Application Priority Data
Jul. 6, 2005 (GB) 0513878.9

(51) Int. Cl. C12Q 1/68; C07H 21/04; G06F 17/30

(52) U.S. Cl. 435/6; 536/24.31; 536/24.33; 536/23.1; 707/758; 707/E17.104

(57) ABSTRACT

The invention relates to a method for determining the level of Fel d1 expression in a cat, the method comprising: a) typing one or more polymorphic positions of the Fel d1 gene in a sample from the cat; and b) thereby determining the level of Fel d1 expression.

### FIELD OF THE INVENTION

The present invention relates to methods for identifying cats susceptible to high or low levels of allergen production and to novel polynucleotides and polypeptides.

### BACKGROUND OF THE INVENTION

Approximately 10% of the worlds human population are allergic to cats and up to 67% of asthmatic patients are sensitive to cat allergens. The major allergen produced by cats is the glycoprotein Fel d1, which elicits a response in 90-95% of patients suffering from cat allergy. This 39 kDa protein is formed from two 17 kDa subunits, each consisting of two disulphide-linked peptides encoded by the genes FEL D1 A and FEL D1 B. The major source of the Fel d1 protein is the sebaceous glands, although expression is also detected in salivary glands and the anal glands. The function of the Fel d1 protein is currently unknown, although it is possibly a pheromone binding protein.

Little is known about the control of Fel d1 expression. Skin production varies depending on anatomical sites, with facial skin producing more protein than the skin on the chest of intact male cats. Fel d1 production falls after neutering and this loss can be reverted by testosterone supplementation. Although Fel d1 is very immunogenic, with potential sensitization being caused by airbourne levels as low as 100 ng/m³, a reduction in the levels of Fel d1 in the environment has been shown to reduce the symptoms of cat allergy sufferers.

### SUMMARY OF THE INVENTION

The present inventors have discovered that the levels of Fel d1 expression vary significantly between cats, with a greater than 7-fold difference between the highest and lowest levels of expression. Identification of cats that produce lower levels of Fel d1 could, therefore, be of advantage to cat allergy sufferers. The inventors have therefore determined the genetic basis of Fel d1 expression in cats in order to provide a method of identifying cats that are likely to have either high or low expression of Fel d1.

Accordingly, the invention provides a method of determining the level of Fel d1 expression in a cat, the method comprising:

a) typing one or more polymorphic positions of the Fel d1 gene in a sample from the cat; and

b) thereby determining the level of Fel d1 expression in the cat.

The invention further provides:

a probe, primer or antibody which is capable of selectively detecting a polymorphic sequence as set out in any one of SEQ ID NOs: 5, 7, 9, 11 and 13;

a kit for carrying out the method of the invention comprising means for detecting the nucleotide present at one or more polymorphic positions of the Fel d1 gene;

a method of providing care recommendations for a cat, the method comprising:

(a) determining the level of Fel d1 expression in the cat by a method of the invention; and

(b) providing appropriate care recommendations to the cat's owner or carrier, and optionally carrying out the care recommendations on the cat.

a method of determining suitability of a cat for an individual who suffers from or is susceptible to Fel d1 allergy, the method comprising:

(a) determining the level of Fel d1 expression in the cat by a method according to any one of claims 1 to 7; and

(b) identifying therefrom the suitability of the cat for the individual;

a database comprising information relating to Fel d1 polymorphisms and their association with levels of Fel d1 expression;

a method for determining the level of Fel d1 expression in a cat, the method comprising:

(a) inputting data of the nucleotide present at one or more polymorphic positions in the cat's Fel d1 gene to a computer system;

(b) comparing the data to a computer database, which database comprises information relating to Fel d1 polymorphisms and their association with levels of Fel d1 expression; and

(c) determining on the basis of the comparison the level of Fel d1 expression in the cat.

a computer program comprising program code means for performing all the steps of a method of the invention when said program is run on a computer;

a computer program product comprising program code means stored on a computer readable medium for performing a method of the invention when said program product is run on a computer;

a computer program product comprising program code means on a carrier wave, which program code means, when executed on a computer system, instruct the computer system to perform a method of the invention;

a computer system arranged to perform a method of the invention comprising:

(a) means for receiving data of the nucleotide present at one or more polymorphic positions in the cat's Fel d1 gene;

(b) a module for comparing the data with a database comprising information relating to Fel d1 polymorphisms and their association with levels of Fel d1 expression; and

(c) means for determining on the basis of said comparison the level of Fel d1 expression in the cat;

an isolated polynucleotide comprising:

(a) a sequence that differs to SEQ ID NO: 1 or 3 at one or more polymorphic positions as defined herein;

(b) any one of SEQ ID NOs: 5,7,9,11 and 13;

(c) a sequence that is complementary or is degenerate as a result of the genetic code to a sequence as defined in (a) or (b); or

(d) a fragment of (a), (b) or (c) which differs to SEQ ID NO: 1 or 3 at one or more polymorphic positions as defined herein and which is at least 10 nucleotides in length;

a polypeptide comprising:

(a) a sequence encoded by a polynucleotide of the invention which differs to SEQ ID NO: 2 or 4 at one or more polymorphic positions as defined herein;

(b) any one of SEQ ID NOs: 6 and 10; or

(c) a fragment of (a) or (b) which differs to SEQ ID NO: 2 or 4 at one or more polymorphic positions as defined herein and which is at least 10 amino acids in length;

a method of selecting a cat for producing offspring with a low or high level of Fel d1 expression comprising:
determining the level of Fel d1 expression by a method according to the invention in a candidate first cat; and thereby determining whether the candidate first cat is suitable for producing offspring with a low or high level of Fel d1 expression, the method further optionally comprising:
determining the level of Fel d1 expression by a method according to the invention in a second cat of the opposite sex to the first cat; and mating the first cat with the second cat in order to produce offspring with a low or high level of Fel d1 expression; and

a method of selecting a cat for desensitising an individual to Fel d1 allergy, comprising:

(a) selecting a cat that has a high level of Fel d1 expression by determining the level of Fel d1 expression according to a method of the invention; and optionally
(b) presenting said cat to a newborn or pregnant human individual.

### BRIEF DESCRIPTION OF THE SEQUENCES

SEQ ID NO: 1 shows the polynucleotide sequence of Fel d1 chain 1. SEQ ID NO: 2 shows the corresponding polypeptide sequence.

SEQ ID NO: 3 shows the polynucleotide sequence of Fel d1 chain 2. SEQ ID NO: 4 shows the corresponding polypeptide sequence.

SEQ ID NOs: 5 and 6 show the polynucleotide and polypeptide sequences of the chain 1 SNP B variant of Fel d1.

SEQ ID NOs: 7 and 8 show the polynucleotide and polypeptide sequences of the chain 1 SNP C variant of Fel d1.

SEQ ID NOs: 9 and 10 show the polynucleotide and polypeptide sequences of the chain 2 SNP A variant of Fel d1.

SEQ ID NOs: 11 and 12 show the polynucleotide and polypeptide sequences of the chain 2 SNP B variant of Fel d1.

SEQ ID NOs: 13 and 14 show the polynucleotide and polypeptide sequences of the chain 2 SNP C variant of Fel d1.

SEQ ID NOs: 15 to 29 show primer sequences.

### BRIEF DESCRIPTION OF THE DRAWINGS

FIG. 1 shows a comparison of the Fel d1 levels extracted from hair samples from 50 cats. The mean value of the 4 samples for each cat is shown. Error bars represent the standard error between these 4 values. The horizontal black line shows the mean value obtained from the results from all 50 cats. The 4 values shaded in darker grey are obtained from cats where at least one of the 4 samples fell out of the standard range of the ELISA.

FIG. 2 shows the final value obtained for Fel d1 production by all 50 cats in relation to their date of birth. Error bars represent the standard error between the 4 samples for each cat. The solid horizontal line represents the overall mean of the values from all 50 cats.

FIG. 3 shows the effect of sex on Fel d1 production. The values for female (pale grey, left hand side) and male (dark grey, right hand side) cats are separated. Error bars represent the standard error between the 4 samples from each cat. Short horizontal lines represent the mean values from all the female cats (left) or all the male cats (right). The long horizontal black line represents the overall mean value from all 50 cats.

FIG. 4 shows the values of Fel d1 extracted from each of the 50 cats plotted against their respective coat colour. Error bars represent the standard error between the 4 samples for each cat. The solid horizontal line represents the overall mean of the values from all 50 cats.

FIG. 5 shows the locations of exons and SNPs in the Fel d1 chain 1 sequence.

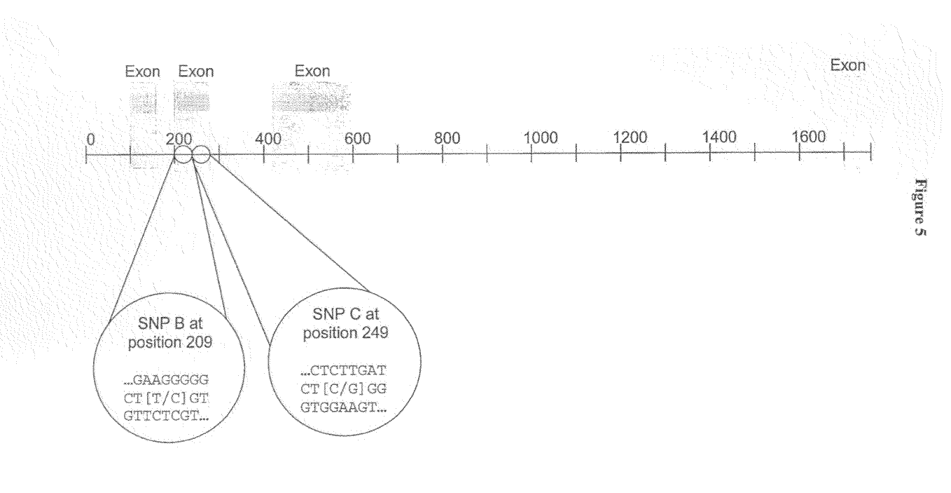

FIG. 6 shows the locations of exons and SNPs in the Fel d1 chain 2 sequence.

FIG. 7 shows the Fel d1 chain 1 sequence including the SNP positions (boxed residues), exon locations (bold) and primer sequences (underlined).

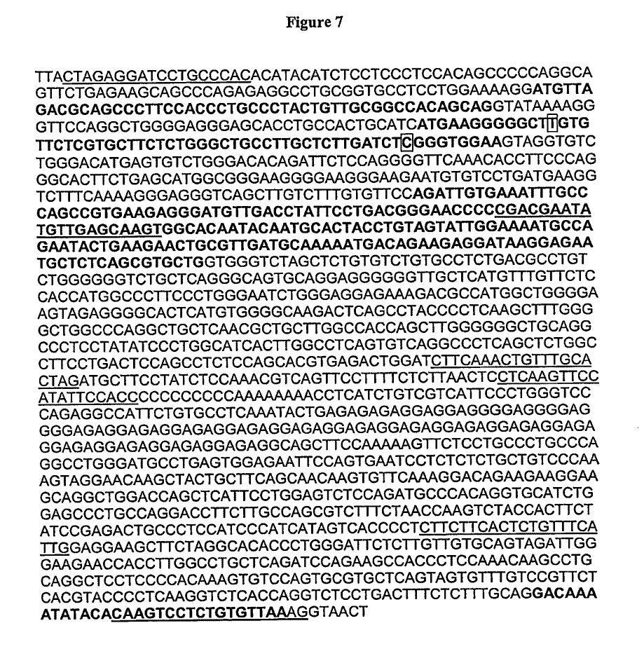

FIG. 8 shows the Fel d1 chain 2 sequence including the SNP positions (boxed residues), exon locations (bold) and primer sequences (underlined).

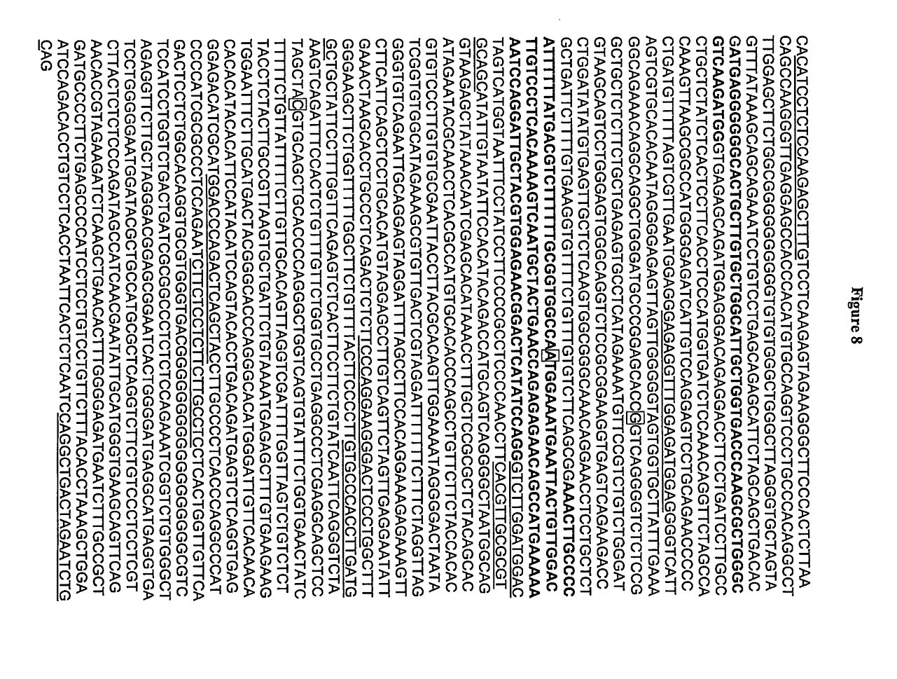

FIG. 9 illustrates schematically an embodiment of functional components arranged to carry out the present invention.

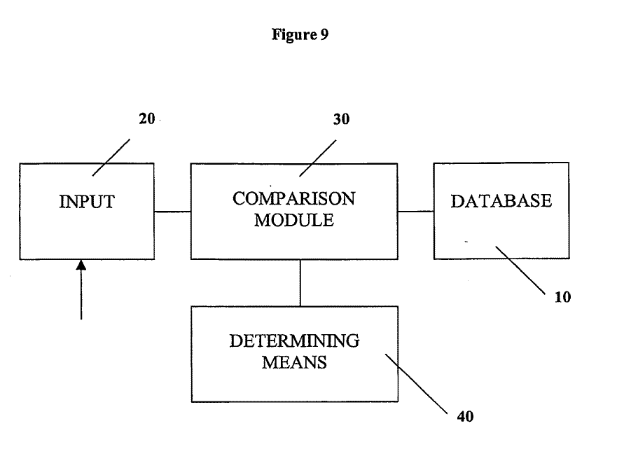

### DETAILED DESCRIPTION OF THE INVENTION

The major cat allergen Fel d1 is composed of two distinct chains, each of which consists of a number of exons. Chain 1 contains exon sequences at positions 102-150 (exon 1), 197-257 (exon 2), 411-600 (exon 3) and 1702-1731 (exon 4). Chain 2 contains exon sequences at positions 215-275 (exon 1) and 791-948 (exon 2).

The present invention allows the identification of cats that are likely to be high or low producers of Fel d1 by detection of polymorphisms, typically in sequences encoding the Fel d1 protein. Preferably, such a polymorphism is at any one of the following positions in relation to the wild-type chain 1 sequence (SEQ ID NO: 1), or is in linkage disequilibrium with a polymorphism at any one of the following positions:

[TIC] at position 209 (chain 1 SNP B); or

[C/G] at position 249 (chain 1 SNP C);

or at any of the following positions in relation to the wild-type chain 2 sequence (SEQ ID NO: 3):

[VG] at position 833 (chain 2 SNP A);

[G/A] at position 570 (chain 2 SNP B); or

[C/T] at position 1620 (chain 2 SNP C).

In each case, the first nucleotide refers to the known (wild-type) Fel d1 gene sequence nucleotide and the second nucleotide refers to the SNP nucleotide.

Two of these SNPs result in coding changes that may be detected in the encoded protein. Chain 1 SNP B (T to C change at position 209) results in a coding change from TTG encoding Leucine (L) to TCG encoding Serine (S). Chain 2 SNP A (A to G change at position 833) results in a coding change from AAT encoding Asparagine (N) to AGT encoding Serine (S). The polymorphic polypeptide sequences are set cut in SEQ ID NO: 6 (chain 1 SNP B) and SEQ ID NO: 10 (chain 2 SNP A).

In one embodiment of the invention, the chain 2 SNP A haplotype GG and the chain 2 SNP C haplotype TT are associated with high levels of Fel d1 expression whereas the chain 2 SNP A haplotype AA and chain 2 SNP C haplotype CC are associated with low levels of Fel d1 expression. In a further embodiment, the gene haplotype CC of the chain 1 SNP B and chain 1 SNP C are associated with high levels of Fel d1 expression and TT/GG are associated with low levels of Fel d1 expression.

The haplotypes of all five SNPs can be expressed in the form: “Chain 1 SNP B/Chain 1 SNP C/Chain 2 SNP A/Chain 2 SNP B/Chain 2 SNP C”. Thus in another embodiment of the invention, the Fel d1 gene haplotypes CT\CG\GG\GG\TT, CT\GG\GG\GG\TT, CT\CC\AG\AG\CT, CC\GG\AG\GG\CT, CC\GG\GG\GG\CT, CC\CG\GG\GG\CC, CC\CG\AA\AG\CC, TT\CC\GG\GG\TT are associated with significantly higher levels of allergen and CC\CG\AG\AG\CC and CT\CG\AG\GG\CT are associated with significantly lower levels.

Polymorphisms which are in linkage disequilibrium with each other in a population tend to be found together on the same chromosome. Typically, one is found at least 30% of the time, for example at least 40%, 50%, 70% or 90%, of the time the other is found on a particular chromosome in individuals in the population. Thus, polymorphisms which are not functional polymorphisms, but are in linkage disequilibrium with the functional polymorphisms, may act as a marker indicating the presence of the functional polymorphism. Polymorphisms which are in linkage disequilibrium with any of the polymorphisms mentioned herein are typically within 500 kb, preferably within 400 kb, 200 kb, 100 kb, 50 kb, 10 kb, 5 kb or 1 kb of the polymorphism. The polymorphism which is detected is typically the functional mutation which contributes to high or low Fel d1 expression, but may be a polymorphism which is in linkage disequilibrium with the functional mutation.

The level of Fel d1 expression in the cat may be equated to the amount of Fel d1 polypeptide detectable on the cat's hair. The term “high level of Fel d1 expression” may be used to describe a level of Fel d1 expression or production that is greater than the mean or median average, and a “low level of Fel d1 expression” may be used to describe a level of Fel d1 expression or production that is smaller than the mean or median average. For example, a high level of Fel d1 expression may correspond to an amount of Fel d1 extracted from a cat hair sample of more than 5000 mU/25mg of hair, such as from 6000 to 16000, from 7000 to 14000, for example about 1000 mU/25mg of hair. A low level of Fel d1 expression of may correspond to an amount of of Fel d1 extracted from a cat hair sample less than 5000 mU/25mg of hair, such as from 1000 to 4000, from 2000 to 3000, in particular about 4000 mU/25 mg of hair. The amount of Fel d1 detectable on the cat's hair may be measured by any suitable means.

### Care Recommendations and Suitability for Owner

Once the level of Fel d1 expression of the cat has been predicted using the method of the invention, it is possible to provide care recommendations appropriate for the cat to the cat's owner or carrier. In particular, if the SNP sequences detected in the cat indicate a high level of Fel d1 expression, the owner or carrier may be taught or shown methods for reducing levels of Fel d1. For example, the care recommendations may comprise instructions for washing and/or brushing the cat to reduce Fel d1 levels. Also, it is known that testosterone causes an increase in Fel d1 production. Therefore, the care recommendations may comprise neutering or castration of a cat, if this has not already been carried out. Once the care recommendations have been provided to the cat's owner or carrier, the care recommendations may optionally be carried out.

The level of Fel d1 expression in a cat may also be used to determine the suitability of the cat for an individual who suffers from or is susceptible to Fel d1 allergy. In particular, a cat that is susceptible to high levels of Fel d1 expression, as determined by typing polymorphic positions in the Fel d1 gene in accordance with the present invention, would not be suitable for an individual who suffers from or is susceptible to Fel d1 allergy, whereas a cat with predicted low levels of Fel d1 expression would be more suitable. Accordingly, it is possible to test a range of cats to determine a cat that is most suitable for an individual. The number of cats that may be tested is at least 1, preferably at least 2, or at least 5, or at least 10.

Selecting Cats with Low or High Fel d1 Expression

In the present invention, it is possible to select a cat for producing offspring with a low or high level of Fel d1 expression. Once the level of Fel d1 expression of a candidate first cat has been predicted using the method of the invention, it is possible to determine whether the candidate first cat is suitable for producing offspring with a low or high level of Fel d1 expression. By determining the level of Fel d1 expression using the method of the invention in a second cat of the opposite sex to the first cat; and mating the first cat with the second cat, it is possible to produce offspring with a low or high level of Fel d1 expression.

Offspring may thus be produced with a low or high level of Fel d1 expression. In one embodiment, offspring with a low level of Fel d1 expression are produced due to inheritance of at least one polymorphism in the Fel d1 gene that causes low expression. At least 2, 3, 4 or 5 or more polymorphisms that cause low expression may be inherited. Accordingly, the first cat may have at least one polymorphism in the Fel d1 gene which causes low Fel d1 expression which polymorphism is not present in the second cat. Similarly, to achieve production of offspring with a high level of Fel d1 expression, the first cat may have at least one polymorphism in the Fel d1 gene which causes high expression which polymorphism is not present in the second cat.

The invention also provides a method of selecting a cat for desensitising an individual to Fel d1 allergy. The result of desensitisation could be an increased tolerance to cat allergen and lower susceptibility to asthma. During development of the human foetus and in infancy the immune system may be desensitised to allergens. According to the invention therefore, a cat may be selected that has a high level of Fel d1 expression by determining the level of Fel d1 expression using the method of the invention. A cat that has a high level of Fel d1 expression may then be presented to a newborn human individual or pregnant human individual. Thus the cat is typically placed in the presence of the individual (for example within 5 or 10 metres of the individual) for at least 2 days, 1 week or 1 month. This time period may be continuous or discontinuous. The cat may be provided to the pregnant human individual at any stage of the pregnancy, from 9 weeks, from 18 weeks, or from 27 weeks of pregnancy until birth. The cat may be presented during the third trimester of pregnancy, which is from 28 weeks of pregnancy until birth. The cat that has a high level of Fel d1 expression may be presented to a human individual whilst the individual is in infancy, such as from birth to 2 years old, birth until 18 months old, birth until 12 months old, or birth until 6 months old.

### Detection of Polymorphisms

The detection of polymorphisms according to the invention may comprise contacting a Fel d1 polynucleotide or protein of the cat with a specific binding agent for a Fel d1 polymorphism and determining whether the agent binds to the polynucleotide or protein, wherein binding of the agent indicates the presence of the polymorphism, and lack of binding of the agent indicates the absence of the polymorphism.

The method is generally carried out in vitro on a sample from the cat. The sample typically comprises a body fluid and/or cells of the cat and may, for example, be obtained using a swab, such as a mouth swab. The sample may be a blood, urine, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the polymorphism.

In the present invention, any one or more methods may comprise determining the presence or absence of one or more Fel d1 polymorphisms in the cat. The polymorphism is typically detected by directly determining the presence of the polymorphic sequence in a polynucleotide or protein of the cat. Such a polynucleotide is typically genomic DNA, mRNA or cDNA. The polymorphism may be detected by any suitable method such as those mentioned below.

A specific binding agent is an agent that binds with preferential or high affinity to the protein or polypeptide having the polymorphism but does not bind or binds with only low affinity to other polypeptides or proteins. The specific binding agent may be a probe or primer. The probe may be a protein (such as an antibody) or an oligonucleotide. The probe may be labelled or may be capable of being labelled indirectly. The binding of the probe to the polynucleotide or protein may be used to immobilise either the probe or the polynucleotide or protein.

Generally in the method, a Fel d1 polymorphism can be detected by determining the binding of the agent to the polymorphic Fel d1 polynucleotide or protein of the cat. However in one embodiment the agent is also able to bind the corresponding wild-type sequence, for example by binding the nucleotides or amino acids which flank the variant position, although the manner of binding to the wild-type sequence will be detectably different to the binding of a polynucleotide or protein containing the polymorphism.

The method may be based on an oligonucleotide ligation assay in which two oligonucleotide probes are used. These probes bind to adjacent areas on the polynucleotide that contains the polymorphism, allowing after binding the two probes to be ligated together by an appropriate ligase enzyme. However the presence of single mismatch within one of the probes may disrupt binding and ligation. Thus ligated probes will only occur with a polynucleotide that contains the polymorphism, and therefore the detection of the ligated product may be used to determine the presence of the polymorphism.

In one embodiment the probe is used in a heteroduplex analysis based system. In such a system when the probe is bound to polynucleotide sequence containing the polymorphism it forms a heteroduplex at the site where the polymorphism occurs and hence does not form a double strand structure. Such a heteroduplex structure can be detected by the use of a single or double strand specific enzyme. Typically the probe is an RNA probe, the heteroduplex region is cleaved using RNAase H and the polymorphism is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the polymorphism, for example a sequence-specific PCR system, and the presence of the polymorphism may be determined by detecting the PCR product. Preferably the region of the primer which is complementary to the polymorphism is at or near the 3′ end of the primer. The presence of the polymorphism may be determined using a fluorescent dye and quenching agent-based PCR assay such as the Taqman PCR detection system.

The specific binding agent may be capable of specifically binding the amino acid sequence encoded by a polymorphic sequence. For example, the agent may be an antibody or antibody fragment. The detection method may be based on an ELISA system. The method may be an RFLP based system. This can be used if the presence of the polymorphism in the polynucleotide creates or destroys a restriction site that is recognised by a restriction enzyme.

The presence of the polymorphism may be determined based on the change that the presence of the polymorphism makes to the mobility of the polynucleotide or protein during gel electrophoresis. In the case of a polynucleotide, single-stranded conformation polymorphism (SSCP) or denaturing gradient gel electrophoresis (DDGE) analysis may be used. In another method of detecting the polymorphism a polynucleotide comprising the polymorphic region is sequenced across the region that contains the polymorphism to determine the presence of the polymorphism.

The presence of the polymorphism may be detected by means of fluorescence resonance energy transfer (FRET). In particular, the polymorphism may be detected by means of a dual hybridisation probe system. This method involves the use of two oligonucleotide probes that are located close to each other and that are complementary to an internal segment of a target polynucleotide of interest, where each of the two probes is labelled with a fluorophore. Any suitable fluorescent label or dye may be used as the fluorophore, such that the emission wavelength of the fluorophore on one probe (the donor) overlaps the excitation wavelength of the fluorophore on the second probe (the acceptor). A typical donor fluorophore is fluorescein (FAM), and typical acceptor fluorophores include Texas red, rhodamine, LC-640, LC-705 and cyanine 5 (Cy5).

In order for fluorescence resonance energy transfer to take place, the two fluorophores need to come into close proximity on hybridisation of both probes to the target. When the donor fluorophore is excited with an appropriate wavelength of light, the emission spectrum energy is transferred to the fluorophore on the acceptor probe resulting in its fluorescence. Therefore, detection of this wavelength of light, during excitation at the wavelength appropriate for the donor fluorophore, indicates hybridisation and close association of the fluorophores on the two probes. Each probe may be labelled with a fluorophore at one end such that the probe located upstream (5′) is labelled at its 3′ end, and the probe located downstream (3′) is labelled at is 5′ end. The gap between the two probes when bound to the target sequence may be from 1 to 20 nucleotides, preferably from 1 to 17 nucleotides, more preferably from 1 to 10 nucleotides, such as a gap of 1, 2, 4, 6, 8 or 10 nucleotides.

The first of the two probes may be designed to bind to a conserved sequence of the gene adjacent to a polymorphism and the second probe may be designed to bind to a region including one or more polymorphisms. Polymorphisms within the sequence of the gene targeted by the second probe can be detected by measuring the change in melting temperature caused by the resulting base mismatches. The extent of the change in the melting temperature will be dependent on the number and base types involved in the nucleotide polymorphisms.

### Polynucleotides

The invention also provides a polynucleotide that comprises a Fel d1 polymorphic sequence. A Fel d1 polymorphic sequence typically differs from the wild-type Fel d1 chain 1 sequence (SEQ ID NO:1) at one or more of the following polymorphic positions:

[T/C] at position 209 (chain 1 SNP B); or

[C/G] at position 249 (chain 1 SNP C);

or at any of the following positions in relation to the wild-type Fel d1 chain 2 polynucleotide sequence (SEQ ID NO: 3):

[A/G] at position 833 (chain 2 SNP A);

[G/A] at position 570 (chain 2 SNP B); or

[C/T] at position 1620 (chain 2 SNP C).

A polynucleotide of the invention preferably comprises the sequence of SEQ ID NO: 5, SEQ ID NO: 7, SEQ 1D NO: 9, SEQ ID NO: 11 or SEQ ID NO: 13 or a fragment thereof which differs to SEQ ID NO: 1 and SEQ ID NO: 3 at one or more polymorphic positions. The polynucleotide is typically at least 10, 15, 20, 30, 50, 100, 200 or 500 bases long, such as at least or up to 1 kb, 10 kb, 100 kb, 1000 kb or more in length. The polynucleotide will typically comprise flanking nucleotides on one or both sides of (5′ or 3′ to) the polymorphism, for example at least 2, 5, 10, 15 or more flanking nucleotides in total or on each side. Typically, the polynucleotide will be at least 95%, preferably at least 99%, even more preferably at least 99.9% identical to the polynucleotide sequences of SEQ ID NO: 5, SEQ ID NO: 7, SEQ ID NO: 9, SEQ ID NO: 11 or SEQ ID NO: 13. Such numbers of substitutions and/or insertions and/or deletions and/or percentage identity may be taken over the entire length of the polynucleotide or over 50, 30, 15, 10 or less flanking nucleotides in total or on each side.

The polynucleotide may be RNA or DNA, including genomic DNA, synthetic DNA or cDNA. The polynucleotide may be single or double stranded. The polynucleotide may comprise synthetic or modified nucleotides, such as methylphosphonate and phosphorothioate backbones or the addition of acridine or polylysine chains at the 3′ and/or 5′ ends of the molecule.

A polynucleotide of the invention may be used as a primer, for example for PCR, or a probe. A polynucleotide or polypeptide of the invention may carry a revealing label. Suitable labels include radioisotopes such as ³²P or ³⁵S, fluorescent labels, enzyme labels or other protein labels such as biotin.

The invention also provides expression vectors that comprise polynucleotides of the invention and are capable of expressing a polypeptide of the invention. Such vectors may also comprise appropriate initiators, promoters, enhancers and other elements such as, for example, polyadenylation signals which may be necessary, and which are positioned in the correct orientation in order to allow for protein expression. Thus the coding sequence in the vector is operably linked to such elements so that they provide for expression of the coding sequence (typically in a cell). The term “operably linked” refers to a juxtaposition wherein the components described are in a relationship permitting them to function in their intended manner.

The vector may be for example plasmid, virus or phage vector. Typically the vector has an origin of replication. The vector may comprise one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter that can be induced in response to heavy metals such as cadmium. Viral promoters such as the SV40 large T antigen promoter or adenovirus promoters may also be used. Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR).

The vector may further include sequences flanking the polynucleotide that give rise to polynucleotides that comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide into the host genome. Replication-defective adenovirus vectors by contrast remain episomal and therefore allow transient expression.

Polynucleotides of the invention may be used as a probe or primer which is capable of selectively binding to an Fel d1 polymorphism. Preferably the probe or primer is capable of selectively binding to the polynucleotide sequence of SEQ ID NO: 5, SEQ ID NO: 7, SEQ ID NO: 9, SEQ ID NO: 11 or SEQ ID NO: 13. The probe or primer more preferably comprises a fragment of a nucleic acid sequence of any one of SEQ ID NO: 5, SEQ ID NO: 7, SEQ ID NO: 9, SEQ ID NO: 11 or SEQ ID NO: 13. The invention thus provides a probe or primer for use in a method according to the invention, which probe or primer is capable of selectively detecting the presence of a Fel d1 polymorphism. Preferably the probe is isolated or recombinant nucleic acid. It may correspond to or be antisense to the polynucleotide sequence of SEQ ID NO: 5, SEQ ID NO: 7, SEQ ID NO: 9, SEQ ID NO: 11 or SEQ ID NO: 13. The probe may be immobilised on an array, such as a polynucleotide array. Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of a full length polynucleotide sequence of the invention.

### Homologues

Homologues of polynucleotide or protein sequences are referred to herein. Such homologues typically have at least 70% homology, preferably at least 80, 90%, 95%, 97% or 99% homology, for example over a region of at least 15, 20, 30, 100 more contiguous nucleotides or amino acids. The homology may be calculated on the basis of nucleotide or amino acid identity (sometimes referred to as “hard homology”).

For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as default a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by at least 1, 2, 5, 10, 20 or more mutations, which may be substitutions, deletions or insertions of nucleotide or amino acids. These mutations may be measured across any of the regions mentioned above in relation to calculating homology. In the case of proteins the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Shorter polypeptide sequences are also within the scope of the invention. For example, a fragment of a polypeptide sequence of the invention is typically at least 10, 15, 20, 30, 40, 50, 60, 70, 80, 100, 150 or 200 amino acids in length. Polypeptides of the invention may be chemically modified, for example post-translationally modified. The polypeptides may be glycosylated or comprise modified amino acid residues. Such modified polypeptides fall within the scope of the term “polypeptide” of the invention.

The polypeptides, polynucleotides, vectors, cells or antibodies of the invention may be present in an isolated or substantially purified form. They may be mixed with carriers or diluents that will not interfere with their intended use and still be regarded as substantially isolated. They may also be in a substantially purified form, in which case they will generally comprise at least 90%, e.g. at least 95%, 98% or 99%, of the proteins, polynucleotides, cells or dry mass of the preparation.

It is understood that any of the above features that relate to polynucleotides and proteins may also be a feature of the other polypeptides and proteins mentioned herein, such as the polypeptides and proteins used in the screening and therapeutic aspects of the invention. In particular such features may be any of the lengths, modifications and vectors forms mentioned above.

### Detector Antibodies

The invention also provides detector antibodies that are specific for a polymorphic polypeptide of the invention. A polymorphic polypeptide of the invention is a polypeptide which differs to the sequence of SEQ ID NO: 2 or SEQ ID NO: 4 at one or more polymorphic positions. Preferably such a polypeptide is encoded by a nucleotide which has a T to C change at position 209 (Fel d1 chain 1 SNP B) and/or by a polynucleotide which has an A to G change at position 833 (Fel d1 chain 2 SNP A). These polymorphisms result in a coding change that is detectable in the polypeptide. More preferably the polypeptide of the invention consists of or comprises the sequence of SEQ 1D NO: 6 or SEQ ID NO: 10.

A detector antibody is an antibody that is specific for one Fel d1 polymorphism but does not bind to any other Fel d1 polymorphism. The detector antibodies of the invention are for example useful in purification, isolation or screening methods involving immunoprecipitation techniques.

Antibodies may be raised against specific epitopes of the polypeptides of the invention. An antibody, or other compound, “specifically binds” to a polypeptide when it binds with preferential or high affinity to the protein for which it is specific but does substantially bind not bind or binds with only low affinity to other polypeptides. A variety of protocols for competitive binding or immunoradiometric assays to determine the specific binding capability of an antibody are well known in the art (see for example Maddox et al, J. Exp. Med. 158 1211-1226, 1993). Such immunoassays typically involve the formation of complexes between the specific protein and its antibody and the measurement of complex formation.

For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample (such as any such sample mentioned herein), which method comprises:

I providing an antibody of the invention;

II incubating a biological sample with said antibody under conditions which allow for the formation of an antibody-antigen complex; and

III determining whether antibody-antigen complex comprising said antibody is formed.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising an antibody in a host animal against the whole polypeptide or a fragment thereof, for example an antigenic epitope thereof, hereinafter the “immunogen”. The fragment may be any of the fragments mentioned herein (typically at least 10 or at least 15 amino acids long).

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

### Detection Kit

The invention also provides a kit that comprises means for typing one or more Fel d1 polymorphism(s) in a cat. In particular, such means may include a specific binding agent, probe, primer, pair or combination of primers, or antibody, including an antibody fragment, as defined herein which is capable of detecting or aiding detection of a polymorphism. The primer or pair or combination of primers may be sequence specific primers that only cause PCR amplification of a polynucleotide sequence comprising the Fel d1 polymorphism to be detected, as discussed herein. The kit may also comprise a specific binding agent, probe, primer, pair or combination of primers, or antibody that is capable of detecting the absence of the polymorphism. The kit may further comprise buffers or aqueous solutions.

The kit may additionally comprise one or more other reagents or instruments that enable any of the embodiments of the method mentioned above to be carried out. Such reagents or instruments may include one or more of the following: a means to detect the binding of the agent to the polymorphism, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the polymorphism as discussed herein, a positive and/or negative control, a gel electrophoresis apparatus, a means to isolate

DNA from sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit typically includes a set of instructions for using the kit.

### Bioinformatics

The sequences of the Fel d1 polymorphisms may be stored in an electronic format, for example in a computer database. Accordingly, the invention provides a database comprising information relating to Fel d1 polymorphic sequences. The database may include further information about the polymorphism, for example the association of the polymorphism with expression of the Fel d1 protein.

A database as described herein may be used to determine the susceptibility of a cat to high or low levels of Fel d1 expression. Such a determination may be carried out by electronic means, for example by using a computer system (such as a PC). Typically, the determination will be carried out by inputting genetic data from the cat to a computer system; comparing the genetic data to a database comprising information relating to Fel d1 polymorphism; and on the basis of this comparison, determining the susceptibility of the cat to high or low levels of Fel d1 expression.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

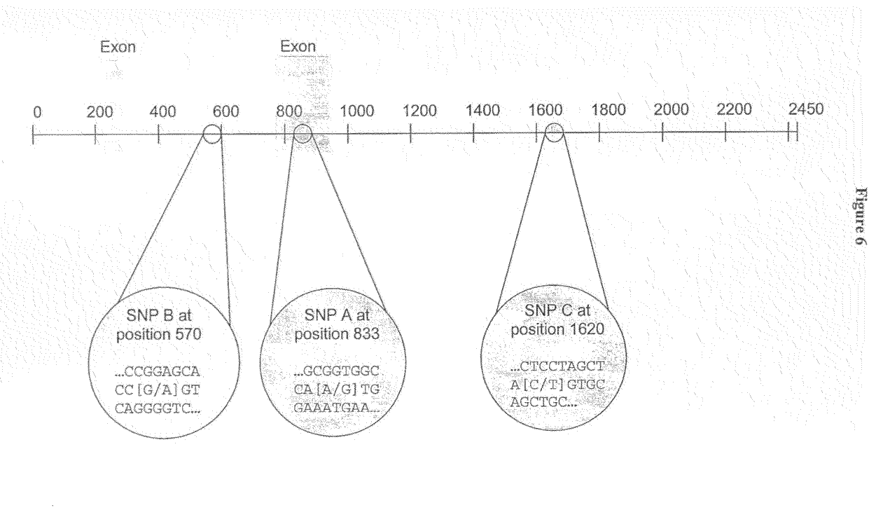

As illustrated in FIG. 6, the invention also provides an apparatus arranged to perform a method according to the invention. The apparatus typically comprises a computer system, such as a PC. In one embodiment, the computer system comprises: means 20 for receiving genetic data from the cat; a module 30 for comparing the data with a database 10 comprising information relating to Fel d1 polymorphisms; and means 40 for determining on the basis of said comparison the cat's level of Fel d1 expression.

The invention is illustrated by the following Examples:

### Example 1

### Materials and Methods

### Samples

A 6 g hair sample was collected by pooling hair from a large number of cats during routine grooming. Hair samples from 50 domestic short-haired cats were obtained by brushing twice a week for 4 consecutive weeks. All cats were either neutered or castrated. Details of all 50 cats are given in Table 1. Each cat was brushed with its own comb, which was stored in a sealed bag when not in use. Hair samples were stored in sealed bags at room temperature before assaying.

### Cat Hair Fel d1 Extraction

The method used to remove Fel d1 from cat hair was based on the method used by Carayol (2000) with modifications. Notably, the extraction buffer was phoshate buffered saline (PBS) containing 0.05% v/v Tween 20, and this was distributed over the hair sample by vortex mixing for 15 minutes. Extraction was carried out in either 15 or 50 ml centrifuge tubes. Any hair that had risen above the surface of the liquid was pressed down beneath the surface and the tubes were incubated at 4° C. for between 16-24 hours. The hair-PBS/Tween mix was then centrifuged at 1900 g for 15 minutes at 4° C. Samples of the supernatant were removed and stored at −20° C. prior to assaying.

TABLE 1
Details of cats tested

| Cat ID number | Sex | Date of birth | Coat colour |
|---|---|---|---|
| H216 | F | 30-Aug-92 | Tabby |
| H219 | F | 10-Sep-92 | Abby Torti |
| H232 | M | 06-Mar-93 | Dark Abby |
| H238 | F | 07-Mar-93 | Tabby |
| H244 | F | 08-Mar-93 | Tabby & White |
| H263 | F | 31-Mar-93 | Tabby & Torti |
| H265 | F | 05-Apr-93 | Tabby |
| H276 | F | 08-Apr-93 | Tabby & White |
| H282 | F | 02-May-93 | Black & White |
| H361 | M | 06-Nov-93 | Tabby |
| H368 | M | 09-Nov-93 | Ginger & White |
| H371 | F | 09-Nov-93 | Tabby & White |
| H389 | F | 08-Apr-94 | Black |
| H414 | F | 20-May-94 | Grey & Cream |
| H427 | F | 24-May-94 | Torti & White |
| H432 | F | 26-May-94 | Tabby & White |
| H519 | F | 25-May-95 | Tabby & White |
| H525 | F | 25-May-95 | Grey & White |
| H553 | M | 03-Aug-95 | Tabby |
| H556 | M | 03-Aug-95 | Grey Tabby |
| H572 | F | 10-Aug-95 | Grey & White |
| H599 | M | 07-Jan-96 | Grey |
| H601 | F | 30-Jan-96 | Tabby |
| H602 | M | 30-Jan-96 | Grey |
| H608 | M | 01-Feb-96 | Grey Tabby |
| H637 | M | 05-Jan-97 | Tabby |
| H645 | M | 15-Jan-97 | Biscuit Ginger |
| H649 | M | 17-Jan-97 | Black |
| H686 | M | 10-Feb-97 | Ginger Stripe |
| H738 | M | 08-Aug-97 | Black |
| H758 | F | 21-Sep-97 | Abby |
| H760 | F | 21-Sep-97 | Abby |
| H774 | M | 08-Oct-97 | Tabby & White |
| H791 | M | 09-Oct-97 | Abby |
| H799 | M | 11-Nov-97 | Pale Ginger |
| H804 | M | 10-Feb-98 | Tabby |
| H812 | M | 08-Mar-98 | Blue & White |
| H826 | M | 02-Apr-98 | Black |
| H827 | M | 02-Apr-98 | Ginger |
| H844 | M | 10-Apr-98 | Tabby & White |
| H858 | F | 29-Apr-98 | Grey Abby |
| H863 | F | 29-Apr-98 | Mackerel Tabby |
| H865 | M | 12-May-98 | Pale Ginger |
| H871 | F | 21-May-98 | Tabby & White |
| H879 | M | 10-Jun-98 | Ginger & White |
| H880 | M | 10-Jun-98 | Tabby & White |
| H883 | M | 11-Jun-98 | Blue Tabby |
| H884 | M | 11-Jun-98 | Grey Tabby |
| H888 | M | 13-May-99 | Ginger & White |
| H900 | M | 27-May-99 | Ginger & White |

Western blotting

Proteins present in the cat hair extract were separated on a polyacrylamide gel, and Fel d1 present in the extract was detected by western blotting using a standard protocol.

### Fel d1 ELISA

The Fel d1 ELISA was carried out using the Indoor Biotechnologies Fel d1 ELISA kit (cat. no. EL-FD1, Indoor Biotechnologies, Cardiff, UK). The manufacturer's instructions were followed except that sample volumes were reduced to 50 μl and tetra-methyl benzidine (Sigma, Poole, UK) was used as the colourimetric dye. The standard curve was created from the Fel d1 standard diluted to between 40 and 0.3125 mU/ml. Hair samples were diluted in extraction buffer before assaying. Data was collected using a Molecular Devices Versamax tuneable microplate reader (Molecular Devices, Wokingham, UK) and analysed using SoftMax Pro software. Results were adjusted for dilution of the hair sample and for different ratios of hair to PBS/Tween used during the extraction process in order to produce a final value of mU Fel d1 extracted per 25 mg of cat hair. For example, the value obtained from a sample extracted at 1 ml/10 mg hair and assayed at a 1/100 dilution was multiplied by 250 (100×2.5) to obtain the final value used.

### Results

### Different Fel d1 Levels Can Be Detected in Different Cats

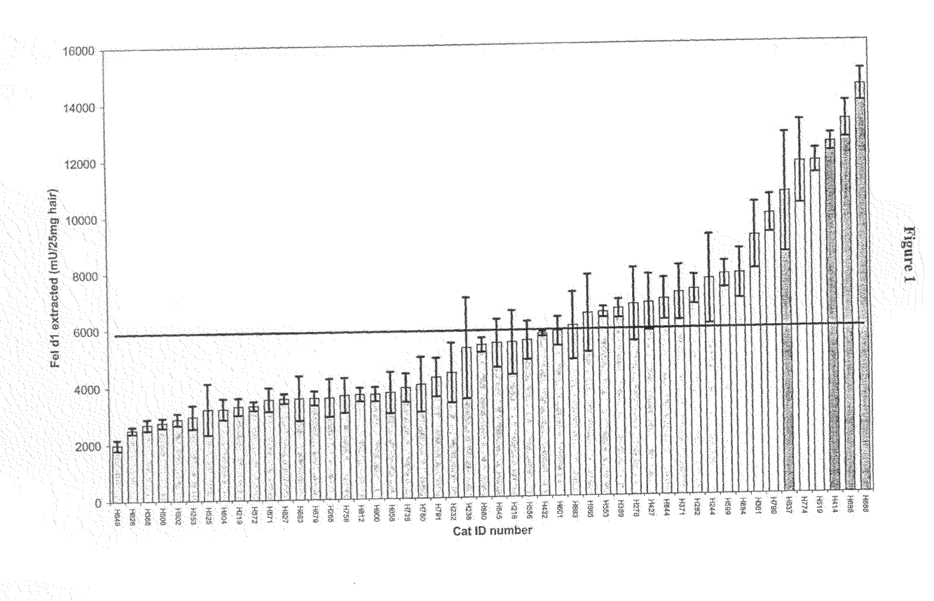

In order to rank cats in terms of their Fel d1 production, 8 hair samples from each cat were combined into 4 pools, each one containing hair from each of the 4 weeks grooming took place. Each sample was assayed in triplicate and the average value for each triplicate was calculated in order to produce a value for each hair extract. This produced 4 Fel d1 values from each cat, one from each of the 4 weeks over which hair sampling occurred. These 4 values were used to calculate a mean value, giving an indication of the Fel d1 production by that particular cat. Results are shown in FIGS. 1 to 4.

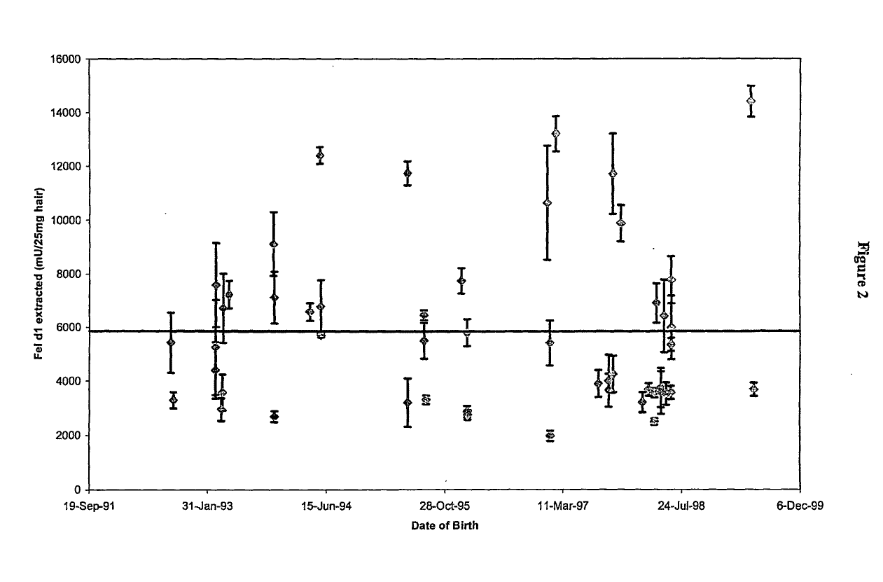

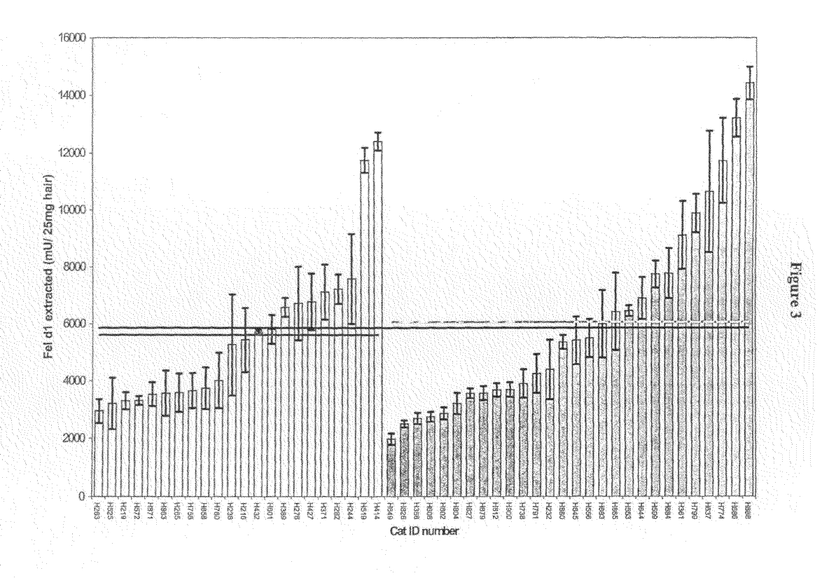

Despite a degree of variation between the 4 samples analysed for each cat, the variation between individual cats was sufficient to enable cats to be ranked in terms of Fel d1 production (FIG. 1). The degree of variation was greater than 7-fold between the lowest and highest producing cats. No obvious relationship between the amount of Fel d1 measured and the cat's age or sex was determined (FIGS. 2 and 3). Although male cats produced more Fel d1 on average than female cats, both sexes contained a wide range of Fel d1 producers, and this difference was not significant. All the male cats used in this study had been neutered and so the increase in Fel d1 production caused by testosterone would not be a contributing factor. Also, the samples were all collected over a four-week period during November and so any seasonal changes or effects of high summer temperatures would not have been detected in these experiments.

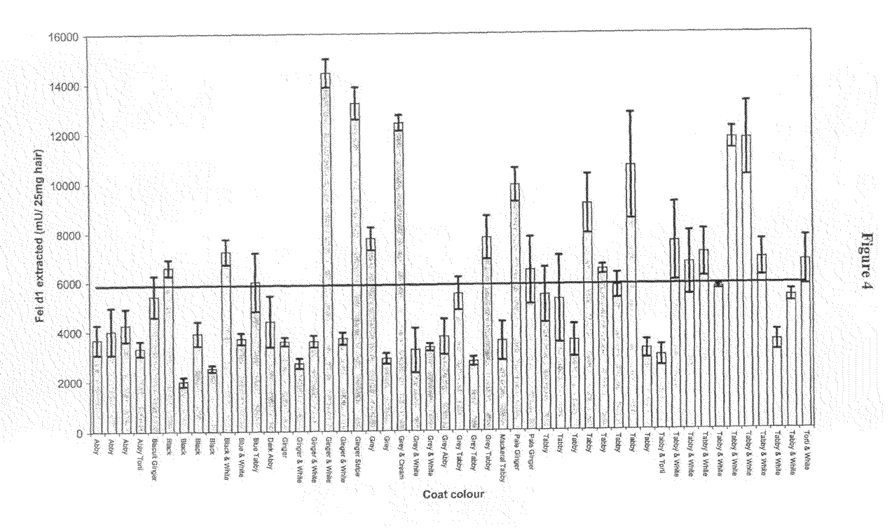

There was a potential correlation between Fel d1 production and some coat colours (FIG. 4). For example, all tabby coloured cats produced a Fel d1 amount below the overall mean and the majority of tabby and white cats produced more Fel d1 than the overall mean. It has been suggested that dark coloured cats produce more Fel d1, but this has been disputed and in these results the black cats in general produced less Fel d1 than the overall average. However, the link between coat colour and Fel d1 production was not statistically significant.

These results show that the amount of Fel d1 on a cat's hair varies between different cats. Since this variation was not related to age, sex or coat colour, the results obtained suggest that this variation may have a genetic basis.

### Example 2

### Materials and Methods

### Identification of SNPs

The Fel d1 gene was resequenced in order to detect single nucleotide polymorphisms (SNPs). Primer sequences are shown in Table 2 below.

TABLE 2

Primer sequences

| | |
|---|---|
| Fel d1 chain 1 primer sequences | Fel d1 chain 2 primer sequences |

| | | |
|---|---|---|
| Forward 1 | 5′ CTAGAGGATCCTGCCCAC 3′ | 5′ CATCCTCTCCAAGAGCTTTG 3′ |
| Reverse 1 | 5′ CGACGAATATGTTGAGCAAGT 3′ | 5′ GAGAGGTTTGGAGATGGAG 3′ |
| Forward 2 | 5′ CTTCAAACTGTTTGCACTAG 3′ | 5′ CCAGGGTCTTGGATGGAC 3′ |
| Reverse 2 | 5′ CTCAAGTTCCATATTCCACC 3′ | 5′ CACGTTGCGCGTGCAGC 3′ |
| Forward 3 | 5′ CTTCTTCACTCTGTTTCATTG 3′ | 5′ CCAGGAAGGGACTCCCTG 3′ |
| Reverse 3 | 5′ CAAGTCCTCTGTGTTAAAG 3′ | 5′ GTGCCCACCTTGATGGC 3′ |
| Forward 4 | | 5′ GGACCCAGACTCAGCTAC 3′ |
| Reverse 4 | | 5′ CTTCTCCTCTTCTTGCCTC 3′ |
| Reverse 5 | | 5′ CAGGCTGACTAGAATCTGC 3′ |

### Analysis of SNPs

In order to investigate whether there is a link between Fel d1 SNPs and level of allergen expression the nucleotide(s) present at each SNP in 49 cats that were tested for allergen levels in Example 1 was determined. Firstly, the effect of each SNP variable was analysed separately using a univariate ANOVA. Table 3 below shows the observed average allergen level associated with each SNP, and the p-value of the test.

TABLE 3
Relationship between SNPs and average level of allergen

| | Average Level of Allergen | | | |
|---|---|---|---|---|
| SNP | AA/CC/CC | AG/CG/CT | GG/GG/TT | P-value |
| Chain 1 SNP B | 5,424 | 7,258 | 6,349 | 0.317 |
| Chain 1 SNP C | 7,531 | 5,445 | 5,614 | 0.505 |
| Chain 2 SNP A | 4,731 | 5,309 | 8,082 | 0.006 |
| Chain 2 SNP B | | 5,408 | 5,725 | 0.770 |
| Chain 2 SNP C | 4,833 | 7,207 | 9,045 | 0.003 |

These results show that individually, chain 2 SNP C is significantly associated with allergen level, with TT having the highest level and CC the lowest. Chain 2 SNP A also has a significant association with GG being associated with the highest level of allergen and AA the lowest. The other SNPs are quite a long way from being significant, with p-values between 0.317 and 0.770.

However, some of these SNPs might be masking/mirroring the action of others. Therefore, a (first order) ‘stepwise’ analysis was carried out. This adds and takes out variables from the model one at a time: at each step it will add the ‘most significant’ unused variable (as long as its p-value, once added, is less than 0.05) or take out the ‘most insignificant’ used variable (as long as its p-value is greater than 0.05). The final model, therefore, should contain the ‘best’ combination of SNPs for predicting allergen level. In the event, the final model contained chain 1 SNP B, chain 1 SNP C and chain 2 SNP C only, and this remained the case whether or not the demographic variables age and sex were included. The final model had an R-sq of 42%. This means that 42% of the variation in the allergen level could be attributed to chain 1 SNP B, chain 1 SNP C and chain 2 SNP C, and all of these SNPs were highly significant (p-values were 0.000 for chain 2 SNP C and chain 1 SNP B, and 0.041 for chain 1 SNP C).

It is also interesting that chain 2 SNP A was significant on its own, but was not so when chain 2 SNP C was put in the model (the p-value of chain 2 SNP A increased from 0.006 to 0.30 when this occurred). This suggests that the values of these two SNPs are probably quite strongly related to each other (i.e. ‘correlated’ in a categorical way). A chi-square test seems to confirm this (p-value=0.000).

### Analysis of Haplotypes

There are 9 haplotypes present in this dataset. Each can be expressed in the form:
“Chain 1 SNP B/Chain 1 SNP C/Chain 2 SNP A/Chain 2 SNP B/Chain 2 SNP C”
   as shown in Table 4 below.

TABLE 4
Haplotype frequencies

| Haplotype | Frequency |
|---|---|
| CC\CG\AA\AG\CC | 1 |
| CC\CG\AG\AG\CC | 2 |
| CC\CG\AG\GG\CC | 2 |
| CC\CG\GG\AG\CC | 1 |
| CC\CG\GG\GG\CC | 2 |
| CC\CG\GG\GG\CT | 1 |
| CC\GG\AA\GG\CC | 15 |
| CC\GG\AG\AG\CC | 1 |
| CC\GG\AG\GG\CC | 10 |
| CC\GG\AG\GG\CT | 4 |
| CC\GG\GG\GG\CT | 2 |
| CT\CC\AG\AG\CT | 1 |
| CT\CG\AG\GG\CT | 3 |
| CT\CG\GG\GG\TT | 1 |
| CT\GG\GG\GG\TT | 1 |
| TT\CC\GG\GG\TT | 2 |

A linear model was run to find whether any of the haplotypes are significantly related to allergen level. It was found that haplotype is significantly related to allergen level (p-value=0.002) and the model has an R-sq value of 61%. Neither age nor sex were significantly related to allergen level, although age was quite close (p-values=0.103 & 0.267 respectively). The structure of significant differences is quite complex, and is represented in the Table 5 below. Membership of ‘homogeneous groups’—which are groups of haplotypes between which there is no significant difference—is indicated by a ‘****” symbol. Therefore if two haplotypes are significantly different none of the four Group columns will contain a ‘*****’ symbol for both.

TABLE 5
Relationship between haplotype and average
allergen level

| | Average | Group | | | |
|---|---|---|---|---|---|
| Haplotype - Full | Allergen Level | 1 | 2 | 3 | 4 |
| CC\CG\AG\AG\CC | 3,038 | **** | | | |
| CT\CG\AG\GG\CT | 3,391 | **** | | | |
| CC\GG\AG\AG\CC * | 3,575 | **** | **** | | |
| CC\CG\AG\GG\CC | 4,501 | **** | **** | | |
| CC\GG\AA\GG\CC | 4,650 | **** | **** | | |
| CC\GG\AG\GG\CC | 4,731 | **** | **** | | |
| CC\CG\GG\GG\CT * | 5,350 | **** | **** | **** | |
| CC\CG\GG\AG\CC * | 5,996 | **** | **** | **** | **** |
| TT\CC\GG\GG\TT | 6,349 | **** | **** | **** | |
| CC\CG\AA\AG\CC * | 6,904 | **** | **** | **** | **** |
| CC\CG\GG\GG\CC | 7,763 | | **** | **** | **** |
| CC\GG\GG\GG\CT | 8,883 | | | **** | **** |
| CC\GG\AG\GG\CT | 9,023 | | | **** | **** |
| CT\CC\AG\AG\CT * | 9,895 | | | **** | **** |
| CT\GG\GG\GG\TT * | 11,730 | | | | **** |
| CT\CG\GG\GG\TT * | 11,754 | | | | **** |

These results show that the highest levels of allergen are associated with the haploptypes CT\CG\GG\GG\TT, CT\GG\GG\GG\TT, CT\CC\AG\AG\CT, CC\GG\AG\GG\CT, CC\GG\GG\GG\CT, CC\CG\GG\GG\CC, CC\CG\AA\AG\CC, TT\CC\GG\GG\TT. The lowest allergen levels were found for CC\CG\AG\AG\CC and CT\CG\AG\GG\CT.

### CONCLUSIONS

The present inventors have discovered single SNPs and SNP haplotypes in the cat Fel d1 gene that are associated with high and low levels of cat allergen production. The detection of these SNPs therefore allows the identification of cats that are likely to be high or low producers of the Fel d1 allergen, and the provision of appropriate care recommendations.

1. A method of determining the level of Fel d1 expression in a cat, the method comprising:
(a) typing one or more polymorphic positions of the Fel d1 gene in a sample from the cat; and
(b) thereby determining the level of Fel d1 expression in the cat.

2. A method according to claim 1,
wherein the one or more polymorphic positions are any of the following:
[T/C] at position 209 of SEQ ID NO: 1 (chain 1 SNP B);
[C/G] at position 249 of SEQ ID NO: 1 (chain 1 SNP C);
[A/G] at position 833 of SEQ ID NO: 3 (chain 2 SNP A);
[G/A] at position 570 of SEQ ID NO: 3 (chain 2 SNP B); or
[C/T] at position 1620 of SEQ ID NO: 3 (chain 2 SNP C); or
a polymorphism which is in linkage disequilibrium with any such polymorphism.

3. A method according to claim 2, wherein the
chain 2 SNP A haplotype GG and the chain 2 SNP C haplotype TT are associated with high levels of Fel d1 expression and the chain 2 SNP A haplotype AA and chain 2 SNP C haplotype CC are associated with low levels of Fel d1 expression.

4. A method according to claim 2, wherein the
gene haplotypes CT\CG\GG\GG\TT, CT\GG\GG\GG\TT, CT\CC\AG\AG\CT, CC\GG\AG\GG\CT, CC\GG\GG\GG\CT, CC\CG\GG\GG\CC, CC\CG\AA\AG\CC, TT\CC\GG\GG\TT are associated with high levels of Fel d1 expression and CC\CG\AG\AG\CC and CT\CG\AG\GG\CT are associated with low levels of Fel d1 expression.

5. A method according to claim 1, wherein step (a) comprises contacting a Fel d1 polynucleotide or protein with a specific binding agent and determining whether the agent binds to the polynucleotide or protein.

6. A method according to claim 5, wherein the agent is a polynucleotide.

7. A method according to claim 1, wherein the nucleotide present at one or more polymorphic positions of the Fel d1 gene is detected by measuring the mobility of a polynucleotide encoding Fel d1 or protein during gel electrophoresis.

8. A probe, primer or antibody which is
capable of selectively detecting a polymorphic sequence as set out in any one of SEQ ID NO:s 5, 7, 9, 11 and 13, optionally in the form of a kit.

9. (canceled)

10. (canceled)

11. A method of providing care recommendations for a cat, the method comprising:
(a) determining the level of Fel d1 expression in the cat by a method according to claim 1; and
(b) providing appropriate care recommendations to the cat's owner or carrier, and optionally carrying out the care recommendations on the cat.

12. A method according to claim 11, wherein the cat
has a high level of Fel d1 expression and the care recommendations comprise instructions for washing and/or brushing the cat to reduce Fel d1 levels.

13. A method according to claim 11, wherein the cat
has a high level of Fel d1 expression and the care recommendations comprise neutering the cat.

14. A method of determining suitability
of a cat for an individual who suffers from or is susceptible to Fel d1 allergy, the method comprising:
(a) determining the level of Fel d1 expression in the cat by a method according to claim 1; and
(b) identifying therefrom the suitability of the cat for the individual.

15. A method according to claim 14, wherein a low
level of Fel d1 expression indicates suitability for an individual who suffers from or is susceptible to Fel d1 allergy.

16. A database comprising information relating to
Fel d1 polymorphisms and their association with levels of Fel d1 expression.

17. A method for determining the level of Fel d1 expression in a cat, the method comprising:
(a) inputting data of the nucleotide present at one or
more polymorphic positions in the cat's Fel d1 gene to a computer system;
(b) comparing the data to a computer database, which database comprises information relating to Fel d1 polymorphisms and their association with levels of Fel d1 expression; and
(c) determining on the basis of the comparison the level of Fel d1 expression in the cat.

18. A method according to claim 17, wherein the Fel d1 polymorphisms are as defined in claim 2.

19. A computer program encoded on a computer-readable medium and comprising program code which, when executed, performs all the steps of claim 17, or a computer system arranged to perform a method according to claim 17 comprising:
(a) means for receiving data of the nucleotide present at one or more polymorphic positions in the cat's Fel d1 gene;
(b) a module for comparing the data with a database comprising information relating to Fel d1 polymorphisms and their association with levels of Fel d1 expression; and
(c) means for determining on the basis of said comparison the level of Fel d1 expression in the cat.

20. (canceled)

21. (canceled)

22. (canceled)

23. An isolated polynucleotide or polypeptide comprising:
(a) a polynucleotide sequence that differs to SEQ ID NO: 1 or 3 at one or more polymorphic positions as defined in claim 2, or a polypeptide sequence encoded by such a polynucleotide which differs to SEQ ID NO: 2 or 4 at one or more polymorphic positions as defined in claim 2;
(b) any one of SEQ ID NO:s 5, 7, 9, 11 and 13 or any one of SEQ ID NO:s 6 and 10;
(c) a polynucleotide sequence that is complementary or is degenerate as a result of the genetic code to a polynucleotide sequence as defined in (a) or (b); or
(d) a polynucleotide fragment of (a), (b) or (c) which differs to SEQ ID NO: 1 or 3 at one or more polymorphic positions as defined in claim 2 and which is at least 10 nucleotides in length, or a polypeptide fragment of (a) or (b) which differs to SEQ ID NO: 2 or 4 at one or more polymorphic positions as defined in claim 2 and which is at least 10 amino acids in length.

24. (canceled)

25. A method of selecting a cat for
producing offspring with a low or high level of Fel d1 expression comprising:
determining the level of Fel d1 expression according to claim 1 in a candidate first cat; and thereby determining whether the candidate first cat is suitable for producing offspring with a low or a high level of Fel d1 expression, the method further optionally comprising:
determining the level of Fel d1 expression according to claim 1 in a second cat of the opposite sex to the first cat; and mating the first cat with the second cat in order to produce offspring with a low or high level of Fel d1 expression.

26. A method according to claim 25 for producing
offspring with a low level of Fel d1 expression, wherein optionally said first cat has at least one polymorphism in the Fel d1 gene which causes low expression which polymorphism is not present in the second cat.

27. A method according to claim 25 for producing offspring with a high level of Fel d1 expression, wherein optionally said first cat has at least one polymorphism in the Fel d1 gene which causes high expression which polymorphism is not present in the second cat.

28. A method of selecting a cat for desensitising an individual to Fel d1 allergy, comprising:
(a) selecting a cat that has a high level of Fel d1 expression by determining the level of Fel d1 expression according to claim 1; and optionally
(b) presenting said cat to a newborn or pregnant human individual.
